(19) United States
(12) Patent Application Publication
(10) Pub. No.: US 2023/0181116 A1
(43) Pub. Date: Jun. 15, 2023

(54) DEVICES AND METHODS FOR SENSING PHYSIOLOGICAL CHARACTERISTICS

(71) Applicant: Huma Therapeutics Limited, London (GB)

(72) Inventors: Sami Alsindi, London (GB); Marko Balabanovic, London (GB); Benjamin Klasmer, London (GB); Sophie Louise Valentine, London (GB); Shahram Nikbakhtian, London (GB); Danoosh Vahdat, London (GB)

(21) Appl. No.: 17/999,294

(22) PCT Filed: May 28, 2021
(86) PCT No.: PCT/GB2021/051318

§ 371 Date: Nov. 18, 2022

(30) Foreign Application Priority Data
May 28, 2020 (GB) 2008043.8

(51) Int. Cl. A61B 5/00; A61B 5/0205; A61B 5/113

(52) U.S. Cl. CPC A61B 5/6898; A61B 5/0205; A61B 5/1135; A61B 5/486; A61B 5/7405; A61B 5/744; A61B 5/7455; A61B 5/7221; A61B 5/02438

(57) ABSTRACT

A device and related method for determining a physiological characteristic of a user, the device including a case having a display disposed on a major face of the case, the case containing a motion sensor configured to measure a signal representative of the physiological characteristic of the user when the case is in contact with the user's body, and a processor configured to receive data characteristic of the signal from the motion sensor, process the data from the motion sensor to determine the physiological characteristic, compare the processed data to at least one of a predetermined threshold or a pattern to determine a quality thereof, and provide feedback to the user to suggest an action by the user to improve a quality of the signal measurement, when the determined quality of the processed data is below a quality associated with the predetermined threshold or pattern.

### RELATED APPLICATION

This application claims priority to and the benefit of GB application number GB 2008043.8 filed May 28, 2020, the disclosure of which is incorporated herein in its entirety.

### FIELD OF THE INVENTION

This invention relates to a device for sensing a physiological parameter such as respiration rate or heart rate. The parameter may be sensed through a mechanism that can provide data when a sensing device, including a motion sensor, is in contact with a user.

### BACKGROUND

The respiratory system of the body facilitates gas exchange. The lungs are the primary organs of the respiratory system. The mouth and nose form the entrance to the airways of the body. The airways include a series of branching tubes, which become narrower, shorter and more numerous as they penetrate deeper into the lung. The primary function of the lung is gas exchange, allowing oxygen to move from the air into the venous blood and carbon dioxide to move out. The trachea divides into right and left main bronchi; the bronchi make up the conducting airways and do not take part in gas exchange. Further divisions of the airways lead to the respiratory bronchioles, and eventually to the alveoli. The alveolated region of the lung is where the gas exchange takes place and is referred to as the respiratory zone.

The process of respiration is divided into two distinct phases: inhalation (inspiration) and exhalation (expiration). During inhalation, the diaphragm contracts and pulls downward while the muscles between the ribs contract and pull upward. This increases the size of the thoracic cavity, thus the pressure inside decreases and air rushes in as a result to fill the lungs. During exhalation, the diaphragm relaxes and the volume of the thoracic cavity decreases, increasing the pressure within it. As a result, the lungs contract and air is expelled.

The main muscles involved in breathing are located in the chest and abdomen. The diaphragm and, to a lesser extent, the intercostal muscles drive respiration during quiet breathing. Additional muscles are typically only used under conditions of high metabolic demand or respiratory dysfunction such as an asthma attack. The diaphragm is a thin, dome-shaped muscle that separates the abdominal cavity from the thoracic cavity. During inhalation, the diaphragm contracts and its centre moves downwards, compressing the abdominal cavity and raising the ribs upward and outward to expand the thoracic cavity. When the diaphragm relaxes, elastic recoil of the thoracic wall causes the thoracic cavity to contract, forcing air out of the lungs and returning to its dome-shape. The intercostal muscles are attached between the ribs and are involved in controlling the width of the rib cage.

A range of respiratory disorders exist. Certain disorders may be characterised by particular events, e.g., apnoeas, hypopnoeas, and hyperpnoeas. Such disorders may be detected and monitored by measuring the respiration rate. Some disorders are associated with an increased or reduced rate of breathing, by an irregular breathing rate or by events such as coughing that disrupt a subject's normal breathing pattern. Some disorders are associated with characteristic noises generated by the respiratory system: for example wheezing or crepitations.

There exist a number of measurement techniques for sensing breathing and calculating respiration rate. An example of such a technique is the use of a belt incorporating a piezoelectric film worn around the chest or abdomen where the breathing of the subject is detected in accordance with a tensile force acting on the belt. Such a configuration in which a belt is worn by the subject, caries the risk that the subject will feel significant discomfort and that the subject's breathing will be affected such that the subject will find it more difficult to breathe while wearing the belt. A further disadvantage of the technique is the limited access of the average household to such equipment.

In an alternative example, a sensor is attached to the chest or abdomen of the subject and the breathing of the subject is detected by detecting changes in the curvature of the chest or abdomen. A problem with this configuration is that the sensitivity with which abdominal breathing is detected is poor when the sensor is attached to the chest of the subject and the sensitivity with which chest breathing is detected is poor when the sensor is attached to the abdomen of the subject. There is also the problem that the breathing sensing device must be large in order to detect changes in curvature.

In a further example, sensors that deform in response to the motion of breathing are adhered to a body. The sensor is configured to detect the breathing of the subject by detecting relative positional changes between the region corresponding to the xiphisternum and the epigastrium. This technique is similarly inaccessible to the majority of users and requires precise positioning of sensors in order to achieve measurements that can be used to generate a reading of respiration rate.

### SUMMARY

There is a need for an improved method of determining breathing or other physiological characteristics using a device that is readily available to a majority of users. A solution to the issue of positioning such a device to detect a suitable signal is also required. It is an object of embodiments of the present invention to provide a physiological characteristic measurement device capable of providing feedback to a user about the optimal position of a detector for measurement.

The physiological characteristic may be a characteristic of a thoracic organ, such as heart rate or respiration rate. The physiological characteristic may be a cyclic physical characteristic.

In an aspect, embodiments of the invention relate to a device for determining a physiological characteristic of a user. The device includes a case including a display disposed on a major face of the case. The case contains a motion sensor configured to measure a signal representative of the physiological characteristic of the user when the case is in contact with the user's body. The case also contains a processor configured to receive data characteristic of the signal from the motion sensor, process the data from the motion sensor to determine the physiological characteristic, compare the processed data to at least one of a predetermined threshold or a pattern to determine a quality thereof, and provide feedback to the user to suggest an action by the user to improve a quality of the signal measurement, when the determined quality of the processed data is below a quality associated with the predetermined threshold or pattern.

One or more of the following features may be included. The physiological characteristic may be a respiratory rate. The respiratory rate may be determined by detecting movement during a plurality of inhalation and exhalation cycles and dividing a number of inhalations or exhalations detected during a period by a duration of the period.

The physiological characteristic may be a heartbeat rate.

The feedback may include instructions to at least one of move the case with respect to the user's body and/or adjust a behavior of the user.

The case may include a smartphone.

The processor may be configured to provide the feedback contemporaneously with the determination of the physiological characteristic to assist placement of the device in contact with the user's body to detect the signal representing the physiological characteristic of the user.

The processor may be configured to generate directions to the user to hold the device on the user's body while the signal is detected.

The motion sensor may include an accelerometer, a gyroscope, a camera, and/or a charge-coupled device.

The feedback may include audio feedback. The physiological characteristic may be a respiratory rate, and the audio feedback may be synchronised with detected inhalation and exhalation of the user. The audio feedback may include vocal cues to ask the user to adjust a position of the case. The audio feedback may indicate the quality of the data characteristic of the signal.

The feedback may include a visual feedback, which may appear on the display. The visual feedback may include a visual representation of the user's body and one or more indicia indicating a position of the device on the user's body and an optimal position for detection. The visual feedback may include video feedback.

The feedback may include a haptic stimuli and/or an optical feedback.

The data may include motion data associated with the rising and falling of the user's chest due to at least one of the user's breathing or the user's heart rate.

The processor may be configured to reject data from the motion sensor when the comparison of the processed data indicates that the user's body was moving during the measurement by the motion sensor, or if no movement is detected.

In another aspect, embodiments of the invention relate to a method for determining a physiological characteristic of a user. The method includes contacting the user's body with a case, the case including a display disposed on a major face of the case. The case contains a motion sensor and a processor. The method further includes measuring, with the motion sensor, a signal representative of the physiological characteristic of the user when the case is in contact with the user's body. Data characteristic of the signal from the motion sensor is received with the processor. The processor processes the data from the motion sensor to determine the physiological characteristic. The processor compares the processed data to at least one of a predetermined threshold or a pattern to determine a quality thereof. The processor provides feedback to the user to suggest an action by the user to improve a quality of the signal measurement, when the determined quality of the processed data is below a quality associated with the predetermined threshold or pattern.

One or more of the following features may be included. The physiological characteristic may be a respiratory rate. Determining the physiological characteristic may include determining the respiratory rate by detecting movement during a plurality of inhalation and exhalation cycles and dividing the number of inhalations or exhalations detected during a period by a duration of the period.

The physiological characteristic may be a heartbeat rate.

The feedback may include instructions to move the case with respect to the user's body and/or adjust a behavior of the user.

The case may include a smartphone.

The feedback may be provided contemporaneously with the determination of the physiological characteristic to assist placement of the device in contact with the user's body to detect the signal representing the physiological characteristic of the user.

The feedback may further include directions to the user to hold the device on the user's body while the signal is detected.

The motion sensor may include an accelerometer, a gyroscope, a camera, and/or a charge-coupled device.

The feedback may include audio feedback.

The physiological characteristic may be a respiratory rate, and the audio feedback may be synchronised with detected inhalation and exhalation of the user.

The audio feedback comprises vocal cues to ask the user to adjust or a position of the case. The audio feedback may indicate the quality of the data characteristic of the signal.

The feedback may include visual feedback. The visual feedback appears on the display. The visual feedback may include a visual representation of the user's body and one or more indicia indicating a position of the device on the user's body and an optimal position for detection. The visual feedback may include video feedback.

The feedback may include a haptic stimuli and/or an optical feedback.

The data may include motion data associated with the rising and falling of the user's chest due to at least one of the user's breathing or the user's heart rate.

The processor may reject data from the motion sensor when the comparison of the processed data indicates that the user's body was moving during the measurement by the motion sensor, or if no movement is detected.

A person different from the user may contact the user's body with the device.

### BRIEF DESCRIPTION OF DRAWINGS

Embodiments of the present invention will now be described by way of example with reference to the accompanying drawings. In the drawings:

FIG. 1 is a schematic illustrating the human respiratory system.

FIG. 2 is a schematic illustrating a physiological sensor device during use on a person, in accordance with an embodiment of the invention.

FIG. 3 is a top view of an exemplary device, in accordance with an embodiment of the invention.

FIG. 4 is a flow chart illustrating an overview of the feedback process during positioning of the device, in accordance with an embodiment of the invention.

FIG. 5 depicts an exemplary visual display on a smartphone of an app for implementing sensing and adjustment, in accordance with an embodiment of the invention.

### DETAILED DESCRIPTION

The following description is presented to enable any person skilled in the art to make and use embodiments of the invention, and is provided in the context of a particular application. Various modifications to the disclosed embodiments will be readily apparent to those skilled in the art.

The general principles defined herein may be applied to other embodiments and applications, without departing from the spirit and scope of embodiments of the present invention. Thus, embodiments of the present invention are not intended to be limited to the embodiments shown, but to be accorded the widest scope consistent with the principles and features disclosed herein.

As used herein, a smartphone is a mobile phone that performs many of the functions of a computer, typically having a touchscreen interface, internet access, and an operating system capable of running downloaded applications.

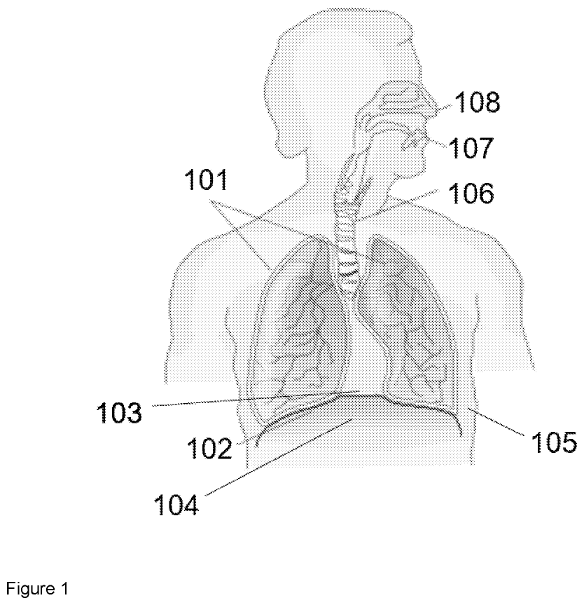

The human respiratory system is shown in FIG. 1. The mouth 107 and nose 108 form the airways that connect to the trachea 106, which branches to meet the lungs 101. The sternum is situated in front of the lungs, with the xiphisternum 103 in the centre of the body. The lungs are supported by the diaphragm 102, which is proximal to the epigastrium 104. Surrounding the lungs at the side of the body are the intercostal muscles 105.

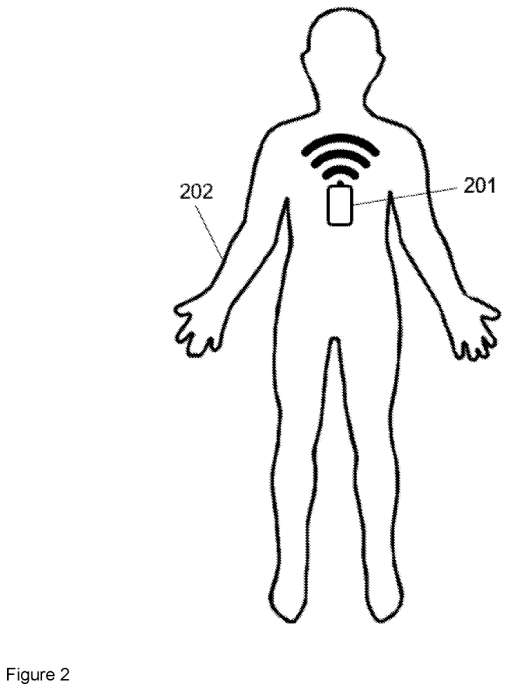

FIG. 2 shows a device 201 positioned in contact with a user's body 202. The device is placed on the user's chest. Alternatively, the device may be held by a user in contact with a body. Conveniently the device may be positioned so as to be adjacent the user's sternum. More generally the device may be positioned so as to be adjacent any part of the user's ribcage or abdomen or adjacent any part of the user's upper chest. Conveniently the device is positioned on the front of the user's body. Conveniently the device is positioned on a part of the user's body that moves as the user breathes in and out. The user may be seated, or prone or standing. Thus, when a major face of a detecting device is placed against the user's chest, that major face may be substantially vertical (e.g., within 20 degrees of vertical) or substantially horizontal (e.g., within 20 degrees of horizontal), or it may be in another orientation. The device may be directly in contact with the user's body (i.e. in contact with the user's skin) or indirectly in contact with the user's body by virtue of being pressed to the user's clothing. The device may be held against a user by that user themselves or by another person. The latter option may be convenient when the first user is, for example, an infant.

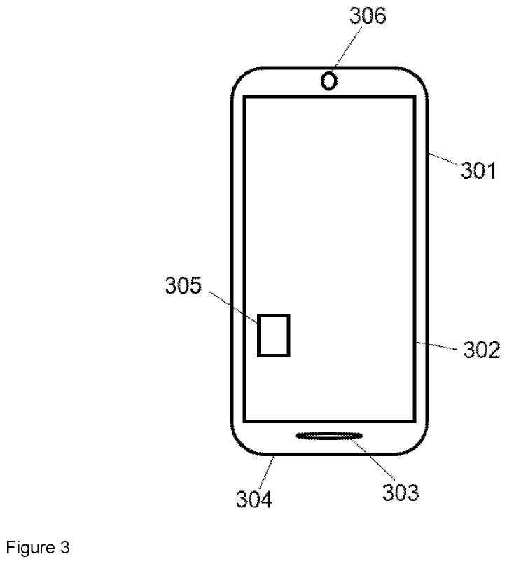

FIG. 3 shows a device suitable as device 201. The device may be a cellular phone, smartphone or tablet. Alternatively, the device may be dedicated for monitoring breathing and/or heart rate. In this example the device 301 of FIG. 3 is a smartphone. The device 301 has a screen 302 for presenting visual feedback to a user. The screen may be an LCD, OLED, LED, plasma or other display; it may be a capacitive or resistive touchscreen that allows a user to input data. The device has one or more sensors 305 present. Each sensor may be a motion sensor, specifically a gyroscope, accelerometer, magnetometer, piezoelectric material, acoustic detector, or equivalent. For example, the device may comprise a multi-axis accelerometer, a magnetometer and a gyroscopic motion sensor. A camera 306 may be provided in the device 301, and the camera may be used to detect motion, i.e., it may be a motion sensor. The device has a microphone 303 that is capable of recording audio inputs. The device further has a speaker 304 that can emit sound, as illustrated in FIG. 2. The device may be configured to connect to an external audio output such as headphones. The device comprises a processor coupled to the screen, sensor(s) 305, camera, microphone and speaker. The processor is also coupled to a transceiver comprised in the device. That may, for example be a transceiver for a wireless protocol. The device comprises a memory that stores in non-transient form code executable by the processor to cause it to perform the functions described herein.

An advantage of the device being a smartphone is that most of the population has access to such a device, meaning that the device enables users to measure their respiration and/or heart rate in domestic settings and at regular intervals to monitor health.

As will be described in more detail below, the processor is capable of:

1. receiving data from the sensor(s) 305 and/or the microphone;

2. analysing that data to attempt to detect artefacts representative of cyclical motion, optionally of cyclical motion whose magnitude and/or frequency and/or variability are in a predetermined range;

3. comparing the result of that detection step with a predetermined set of thresholds or patterns so as to assess whether the data is of a quality associated with those thresholds or patterns; and

4. in dependence on that comparison step, causing one or more of the speaker and the display to provide feedback to the user for encouraging the user to move the device on the user's body so as to improve the quality of the sensed data.

The quality of the detected signal as a means of capturing data about a periodic physiological function may be estimated in any suitable way. For example, in a first step, the magnitude of rotation or translation over segments of the captured data can be measured and compared, to check if the device is being held sufficiently still. In a second step, an estimated frequency of the physiological function may be determined. This may be determined from historic data for typical individuals or by spectral analysis of the captured data to determine one or more dominant frequencies in the data. In a third step an autocorrelation operation may be performed in which the correlation is determined between (i) the captured data and (ii) a copy of the captured data delayed by the period of the frequency determined in the first step. The third step may be repeated for a set of frequencies. In some embodiments, the quality of the captured data may be represented by the uniformity and frequency of the highest values of these autocorrelations.

The output of the detection step may represent a measurement of a physiological parameter of the user such as the user's respiration or heart rate. That output may be stored, presented to the user and/or transmitted to a remote location for further analysis.

The device may estimate a user's tidal volume. This may be estimated from one or more of the following inputs: (i) information regarding the status of the user: for example the user's age, height and/or weight; (ii) an estimate of the user's respiration rate formed in the manner described herein; (iii) information collected by a microphone sensor of the device representing the sound of the user breathing. This data may be combined using a suitable algorithm, for example one derived from machine learning, to estimate the user's tidal volume.

The device comprises a rigid or semi-rigid case that holds the other components described above, e.g., the sensor(s) and processor. The case may be of a cuboid form. Conveniently at least one face of the cuboid has an area greater than 30 cm2. Conveniently two opposite faces of the cuboid have areas that are more than 5 times those of the other faces. These features can facilitate the device being placed flat on a user's chest. When the device has a display the display may be on such a major face.

When the device is in position on the user's chest it may be held against the chest by gravity and/or by a strap, or the user may press the device against the user's chest using his or her hand. The user may be seated or standing. Alternatively the user may lie with his or her chest on the device, preferably over a resilient substrate such as a mattress, or the user may lie on his or her back with the device placed on the chest facing upwards.

To initiate sensing, the user may operate a user interface of the device (e.g., its touchscreen or using voice input) to activate a sensing mode. That sensing mode may be provided by an app or application running on the device. The app may cause the device to display instructions to the user to position the device on the user's chest.

When the sensing mode is initiated, the processor receives data from its inputs (e.g., the motion sensor(s) and/or the microphone) and analyses it to attempt to detect artefacts in the data that are associated with a generally cyclical pattern characteristic of the motion of the chest during respiration. This may, for example, be done by filtering the received data and applying a Fourier transform to it, or applying autocorrelation analysis, or using wavelet functions, or through a trained machine learning algorithm. It may be expected that a given physiological mechanism will have a frequency within known bounds. For example, including cases where an individual is unwell or is tested after exercising, a breathing rate might be in the range from 4 to 50 per minute and a heart rate might be in the range from 20 to 250 per minute. The processor may filter the data to reject data associated with frequencies outside a predetermined band, e.g., if the user's body was moving during the measurement by the motion sensor, or if no movement is detected at all (e.g., if the device is resting on a desk).

When the processor is processing data from the motion sensor(s), it may be configured to identify motion data associated with the rising and falling of the user's chest due to breathing or to the user's heart rate. For that reason, it may give a greater weight to motion data representing translation having a component perpendicular to a major face of the device or rotation having a component in a major plane of the device than to other motions.

The processor may combine data from the motion sensor(s) and the microphone by attributing a greater degree of quality to a sensed frequency if the same frequency is detected from the data from the motion sensor(s) and the data from the microphone.

The processor attributes a quality level to the estimation of a frequency from the sensed data. This may be done in any of a number of ways. The quality may be dependent on any one or more of: (i) the magnitude of a cyclical signal detected in motion and/or audio data, with a greater magnitude indicating greater quality; (ii) the level of agreement in the frequencies of cyclical signals detected from two different sensors (e.g., a motion sensor and the microphone); (iii) the extent to which the strongest detected frequency has a greater strength than the sum of the remaining detected frequencies; (iv) the variability of a cyclical signal over the period of measurement, with a lower variability indicating greater quality and (v) the overall movement of the device (in terms of acceleration, translation or rotation), with too great a movement representing lower quality. A predetermined algorithm may be used to combine any two or more metrics to form an overall quality. A predetermined algorithm may combine metrics that have the highest computed confidence. The algorithm may vary or use different processing steps depending on the specific device or motion sensor used. For example, a specific brand of smartphone or motion sensor may be calibrated differently or produce a different format or resolution or frequency of data. A single selected quality or the overall quality can then be compared with a predetermined threshold or pattern to provide an indication of whether the sensed data is adequate.

If the sensed data is determined not to be adequate then the device provides the user with an output to encourage the user to move the device on the user's body or hold a more still position. The sensed data may be inadequate for a number of reasons: holding the device in the wrong position, moving around during the measurement, holding the device in the wrong orientation, usage of a case around the device that muffles the signals, wearing too much heavy clothing, or not holding the device in position for long enough. Feedback may also indicate that the device is broken and/or the sensors are not suitable, e.g., “there is an error, please try again on a different device or contact our support team.”

That output may be on the device's display. In some embodiments, the output is an audio output from the device's loudspeaker. This has the advantage that it can be better appreciated by the user when the device is positioned on the user's chest. An audio feedback output may take any convenient form. In one example it may be a beep, tone, series of tones, melody or other predetermined non-verbal noise. In another example it may be a verbal output, for example a phrase asking the user to move the device or make other adjustments. In another example it may be a sound generated in dependence on the sensed data. That sound may be a synthesised breathing or heartbeat sound that varies at the same frequency as a cyclical signal that has been detected in the sensed data. The synthesised breathing or heartbeat sound may be in phase with the cyclical signal. The pitch and/or volume of the synthesised sound may be dependent on the quality the sensed data has been estimated to have.

Optionally, the device may receive inputs about the user such as age, height, weight, and pre-existing conditions in order to calibrate the expected output.

The device may be configured to provide feedback to the user for indicating to the user that the position of the device should be varied to improve the quality of the measurement. Such feedback may be provided as at least one of audio, visual or haptic feedback. The audio feedback may be verbal directions such as “move the phone towards your head”, or sound effects representative of proximity to an optimal position such a beeps of varying pitch, frequency or intensity. There may also be feedback to indicate to the user that the phone is positioned correctly. Such feedback may be by any suitable mechanism: for example audio, haptic or visual.

The device may be held by a person different from the user, to contact the user's body with the device. Accordingly, the person holding the device may respond to feedback about device placement. For example, a parent or carer may measure the respiratory rate or heartbeat rate of a child or a baby, or a relative or carer may measure the respiratory rate or heartbeat of an older person.

The accurate measurement of respiratory or heart rate may require a period of measurement such as 30 seconds or 1 minute. However, the device may be configured to provide feedback to the user more frequently, for example after 10 seconds or 20 seconds of measurement. The device may be further configured to provide live and real-time feedback to the user during the course of the measurement, by constantly sampling data quality from preceding windows of 10 seconds or 20 seconds.

The device contains a processor or processing unit that may be configured to distinguish outputs from the sensor caused by the breathing of a user from other movements, other vibrations caused by sounds. Such signal processing may use a frequency filter to distinguish between frequency components, allowing a plurality of signals to be detected simultaneously. The signal caused by the user's breathing may be distinguished from sound signals and the two signals can be compared to detect abnormal breathing such as an asthma attack.

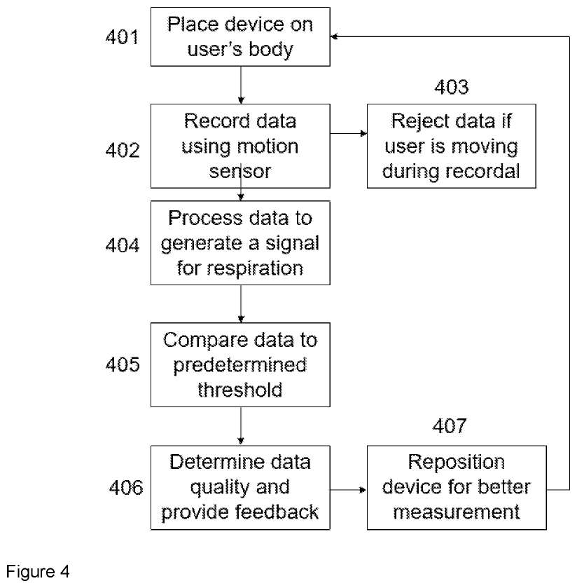

FIG. 4 shows an exemplary process flow for an embodiment of the device. The device is first positioned on a user at step 401. Data is recorded using the motion sensor at step 402, this data is then either rejected at step 403 or processed to generate a signal for separation at step 404. The generated signal is compared with a predetermined threshold to determine the quality of the data at step 405. Dependent on the quality of the data, feedback is provided about the measurement position of the device at step 406, the feedback prompts the user to reposition the device for better measurement at step 407, returning to step 401 to iterate the process again.

The device may be used to detect any one or more of respiration rate, heart rate, tidal volume and other cyclical physiological events or characteristics.

The device may provide a general instruction to a user, indicating for example that the device is to be moved. Alternatively it may provide more specific instructions: for example to move the device to a different position, for the user to stop moving his or her body, for the user to hold the device still for a longer period, for the user to not talk, or for the user to attempt again without coughs, sneezes or sudden movements.

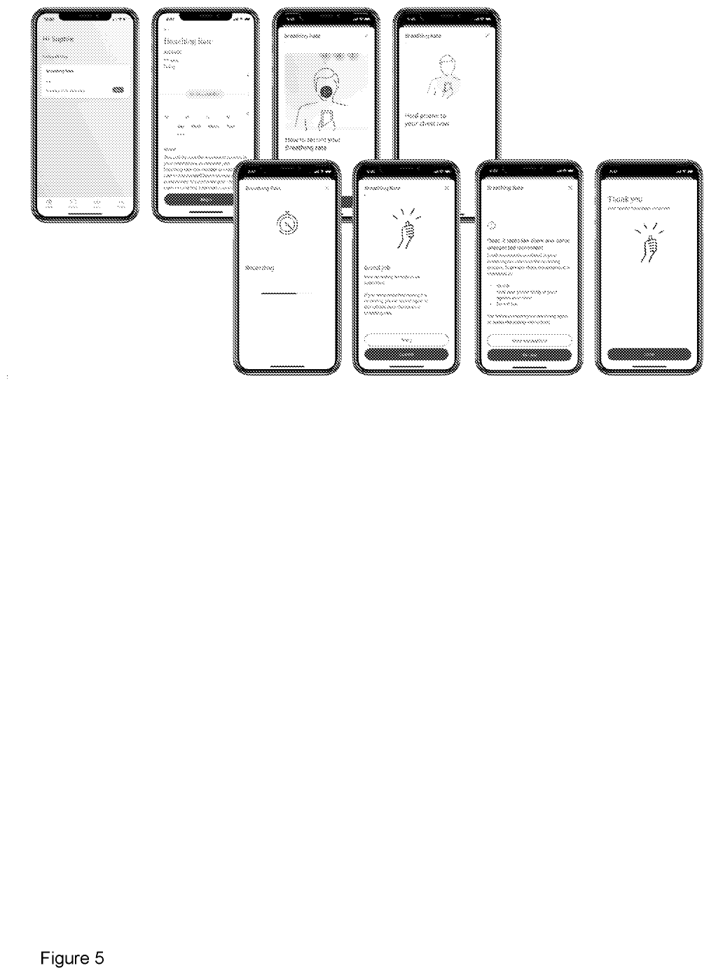

An example of an app on a device configured to perform the assessment is shown in FIG. 5. Various screens of a visual output are shown that provide instructions to a user, such as instruct the user to get ready, record, and hold the phone to the user's chest. The device may also indicate that recording is taking place and that data is being uploaded.

Applications of the device include assessment of health conditions and breath training for relaxation. For example, an indicator of a possible health condition in a subject or an increase in severity of a health condition in a subject may be an increase in the breathing rate of the subject.

### Clinical Safety Features

The device may contain two safety features to facilitate ensuring that the breathing rate signal captured by the user is representative of the user's true breathing rate.

These may be implemented to reduce the risk that breathing rate values derived from the device that are not representative of the user's true breathing rate may be used to inform any action that may impact the health and wellbeing of the user, whether the action is taken by the user, a healthcare professional or any other party. These safety features include:
Messaging presented to the user via a smartphone application interface before a breathing rate recording is initiated providing the user with instructions to follow before and during the breathing rate recording to ensure that the user's breathing rate during the time of recording is maximally representative of the user's true breathing rate. Specifically, these include that the user should rest for a few minutes before initiating the recording and sit upright with his or her legs crossed during the recording.
Messaging presented to the user via the smartphone application interface after a breathing rate recording is taken and before a breathing rate estimate has been generated providing the user with an opportunity to retake the recording if the user believes that his or her breathing rate during the period of the breathing rate recording was not representative of the user's true breathing rate. Specifically, this includes if the user was disturbed during the recording in a way that may have elevated his or her breathing rate.

### Data Use Features

Breathing rate data generated by the device may be used to generate value for individuals or parties involved in the use of the device. Specifically
The user of the device may be presented with the user's breathing rate, as estimated by the device, and may use this to understand, monitor or track the user's breathing rate, including over time, for the purposes of understanding or improving the user's health. The user may additionally be presented with educational resources to help the user understand how his or her breathing rate may impact or act as an indicator of his or her overall health, and actions that can be taken to improve his or her breathing rate or overall health, as indicated by the user's breathing rate.
An individual or party who is not the user of the device, including healthcare professionals, researchers, carers, insurance companies and other individuals or parties with an interest in understanding the breathing rate of an individual, may be presented with the breathing rate of the user of the device. This information may be used by them to manage the healthcare of the user of the device, facilitate participation of the user of the device in a research or clinical trial, understand the health of the user of the device for the purposes of determining health insurance premiums, or other purposes.

Embodiments of the invention may include any individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. Aspects of the present invention may include any such individual feature or combination of features. In view of the foregoing description it will be evident to a person skilled in the art that various modifications may be made within the scope of the invention.

1. A device for determining a physiological characteristic of a user, the device comprising:
a case comprising a display disposed on a major face of the case, the case containing:
a motion sensor configured to measure a signal representative of the physiological characteristic of the user when the case is in contact with the user's body; and
a processor configured to:
receive data corresponding to the signal from the motion sensor,
process the data from the motion sensor to determine the physiological characteristic,
compare the processed data to at least one of a predetermined threshold or a pattern to determine a quality thereof, and
in response to a determination that the quality of the processed data is below a quality associated with the predetermined threshold or pattern, provide feedback to the user to suggest an action by the user to improve a quality of the signal.

2. The device of claim 1, wherein the physiological characteristic is a respiratory rate.

3. The device of claim 2, wherein the respiratory rate is determined by detecting movement during a plurality of inhalation and exhalation cycles and dividing a number of inhalations or exhalations detected during a period by a duration of the period.

4. The device of claim 1, wherein the physiological characteristic is a heartbeat rate.

5. The device of claim 1, wherein the feedback comprises at least one of instructions to move the case with respect to the user's body or instructions to adjust a behavior of the user.

6. The device of claim 1, wherein the case comprises a smartphone.

7. The device of claim 1, wherein the processor is configured to provide the feedback contemporaneously with the determination of the physiological characteristic to assist placement of the device in contact with the user's body to detect the signal representing the physiological characteristic of the user.

8. The device of claim 1 wherein the processor is configured to generate directions to the user to hold the device on the user's body while the signal is detected.

9. The device of claim 1, wherein the motion sensor comprises at least one of an accelerometer, a gyroscope, a camera, or a charge-coupled device.

10. The device of claim 1, wherein the feedback comprises audio feedback.

11. The device of claim 10, wherein the physiological characteristic is a respiratory rate and the audio feedback is synchronized with detected inhalation and exhalation of the user.

12. The device of claim 10, wherein the audio feedback comprises vocal cues to prompt the user to adjust a position of the case.

13. The device of claims 10, wherein the audio feedback indicates the quality of the data characteristic of the signal.

14. The device of claim 1, wherein the feedback comprises a visual feedback.

15. The device of claim 14, wherein the visual feedback appears on the display.

16. The device of claim 15, wherein the visual feedback comprises a visual representation of the user's body and one or more indicia indicating a position of the device on the user's body and an optimal position for detection.

17. The device of claim 14, wherein the visual feedback comprises video feedback.

18. The device of claim 1, wherein the feedback comprises a haptic stimuli.

19. The device of claim 1, wherein the feedback comprises an optical feedback.

20. The device of claim 1, wherein the data comprises motion data associated with the rising and falling of the user's chest due to at least one of the user's breathing or the user's heart rate.

21. The device of claim 1, wherein the processor is configured to reject data from the motion sensor when the comparison of the processed data indicates at least one of (i) the user's body was moving during the measurement by the motion sensor, or (ii) no movement is detected.

22. A method for determining a physiological characteristic of a user, the method comprising:
contacting the user's body with a case, the case comprising a display disposed on a major face of the case, a motion sensor, and a processor;
measuring, with the motion sensor, a signal representative of the physiological characteristic of the user when the case is in contact with the user's body;
receiving, with the processor, data corresponding to the signal from the motion sensor;
processing, with the processor, the data from the motion sensor to determine the physiological characteristic;
comparing, with the processor, the processed data to at least one of a predetermined threshold or a pattern to determine a quality thereof; and
in response to a determination that the quality of the processed data is below a quality associated with the predetermined threshold or pattern, providing feedback, with the processor, to the user to suggest an action by the user to improve a quality of the signal.

23. The method of claim 22, wherein the physiological characteristic is a respiratory rate.

24. The method of claim 22, wherein determining the physiological characteristic comprises determining the respiratory rate by detecting movement during a plurality of inhalation and exhalation cycles and dividing a number of inhalations or exhalations detected during a period by a duration of the period.

25. The method of claim 22, wherein the physiological characteristic is a heartbeat rate.

26. The method of claim 22, wherein the feedback comprises at least one of instructions to move the case with respect to the user's body or instructions to adjust a behavior of the user.

27. The method of claim 22, wherein the case comprises a smartphone.

28. The method of claim 22, wherein the feedback is provided contemporaneously with the determination of the physiological characteristic to assist placement of the device in contact with the user's body to detect the signal representing the physiological characteristic of the user.

29. The method of claim 22, wherein the feedback further comprises directions to the user to hold the device on the user's body while the signal is detected.

30. The method of claim 22, wherein the motion sensor comprises at least one of an accelerometer, a gyroscope, a camera, or a charge-coupled device.

31. The method of claim 22, wherein the feedback comprises audio feedback.

32. The method of claim 22, the physiological characteristic is a respiratory rate, and the audio feedback is synchronized with detected inhalation and exhalation of the user.

33. The method of claim 32, wherein the audio feedback comprises vocal cues to prompt the user to adjust a position of the case.

34. The method of claims 22, wherein the audio feedback indicates the quality of the data characteristic of the signal.

35. The method of claim 22, wherein the feedback comprises a visual feedback.

36. The method of claim 35, wherein the visual feedback appears on the display.

37. The method of claim 36, wherein the visual feedback comprises a visual representation of the user's body and one or more indicia indicating a position of the device on the user's body and an optimal position for detection.

38. The method of claim 35, wherein the visual feedback comprises video feedback.

39. The method of claim 22, wherein the feedback comprises a haptic stimuli.

40. The method of claim 22, wherein the feedback comprises an optical feedback.

41. The method of claim 22, wherein the data comprises motion data associated with the rising and falling of the user's chest due to at least one of the user's breathing or the user's heart rate.

42. The method of claim 22, further comprising rejecting data from the motion sensor, with the processor, when the comparison of the processed data indicates at least one of (i) that the user's body was moving during the measurement by the motion sensor, or (ii) no movement is detected.

43. The method of claim 22, wherein a person different from the user contacts the user's body with the device.
